(19) United States
(12) Patent Application Publication
(10) Pub. No.: US 2022/0162533 A1
(43) Pub. Date: May 26, 2022

(54) CULTURE SUBSTRATE FOR METHANISATION METHOD

(71) Applicant: BioRenGaz, Strasbourg (FR)

(72) Inventor: Jonathan FRITSCH, Strasbourg (FR)

(73) Assignee: BioRenGaz, Strasbourg (FR)

(21) Appl. No.: 17/430,820

(22) PCT Filed: Feb. 12, 2020
(86) PCT No.: PCT/EP2020/053632

§ 371 Date: Aug. 13, 2021

(30) Foreign Application Priority Data
Feb. 15, 2019 (FR) FR1901540

(51) Int. Cl. C12M 1/107; C12M 1/12; C12M 1/00; B01D 53/85

(52) U.S. Cl. CPC C12M 21/04; C12M 25/14; B01D 2258/05; C12M 47/18; B01D 53/85; C12M 29/18

(57) ABSTRACT

A methanisation unit includes a culture substrate to be used in a method for methanising liquid effluents with structured packing, the culture substrate being made up of more than 50% of wood elements, of which at least one dimension is greater than 80 mm, the porosity of the culture substrate being greater than 50%. Embodiments relate to a method for preparing a culture substrate intended to be used in a methanisation unit according to the invention, and a methanisation method in a methanisation unit.

The present invention relates to the field of waste reclamation. It relates more particularly to a culture substrate for a methanisation method.

Methanisation is the natural biological process of degradation of organic material in the absence of oxygen.

This degradation produces a biogas, that can be used as a source of energy. Methanisation is also called anaerobic digestion, which can comprise a step of degradation of biological materials into CH₄ and CO₂ and/or a step of transformation of CO₂ and H₂ into CH₄.

Conventional infinitely mixed liquid methanisation (or CSTR, “Continuous Stirred Tank Reactor”) is currently the most widespread method of methanisation. The latter has the advantage having substantial feedback and the capacity of incorporating a large variability of inputs, but has only low performance per volume unit.

Liquid methanisation with a granular sludge bed (EGSB, “Expanded Granular Sludge Bed” or UASB, “Up-flow Anaerobic Sludge Blanket”) implement reactors wherein the sludges or the granules of bacteria are put into suspension by a recirculation of the medium. These are high-performance compact technologies allowing for the retention of biomass and that treat liquid effluents without matter in suspension. The performance of these methods would be negatively impacted by the presence of suspended materials (SM) and of fatty substances present in the biowaste pulp.

Liquid methanisations equipped with fixed or mobile culture substrates have high treatment yields for liquid effluents and provide increased resistance to inhibitors but are also subjected to different constraints which reduce their performance. Fixed culture substrates are frequently obstructed or clogged by biological and/or mineral deposits requiring cleaning operations or replacement inside digesters.

Mobile culture substrates also known this phenomenon that reduces their performance by decreasing the specific surface and are also subjected to an abrasion that deteriorates them and induces a limited service life. In the two cases the cleaning and/or replacement operations induce substantial production losses as well as tedious maintenance operations and this even before addressing the very high cost of these culture substrates when it is a question of outfitting high volumes. It is common that the culture substrates used for the methanisation be made of plastic material. Such solutions are expensive, coming from fossil resources, and generate plastic waste that is harmful for the environment.

Continuous dry methanisation could make it possible to integrate the culture substrate simultaneously with the biowaste, but in practice, feedback shows substantial energy consumption and many machine breakage problems (blenders, stirrer, pumps, etc.) blocking the operation of the digester. The accumulation of sediments (stones, sand, undesirable items) inside digesters reduces performance and requires tedious curative maintenance operations that require shutting down the affected digester and which generate substantial operating losses.

Discontinuous dry methanisation, wherein the tank is supplied at the loader with gross solids in tunnels/garages, is another existing method. The absence of a stirring and/or mixing system in the tunnels avoids the aforementioned mechanical constraints (breakage, premature wear). However the absence of mechanical pre-treatment (grinding) of the materials treated substantially slows down the degradation kinetics, minimising performance. The rheology problems linked to insufficient and/or non-homogeneous porosity of the material incorporated frequently disturbs the reaction balances and the optimum operation of the installations.

It is common that the culture substrates used for the methanisation be made of plastic material. Such solutions are expensive, and generate plastic waste that is harmful for the environment.

Document EP1818314 discloses a method and biogas production installation from liquids charged with organic material, using a culture substrate of wood chips. However the driving of such a substrate by the suspended materials of the liquid treated causes the agglomeration thereof, then the occlusion thereof and does not make it possible to obtain effective methanisation over time.

An object of the present invention is to propose a culture substrate for methanisation that allows for fast, effective and sustainable methanisation of liquid effluents.

Another object of the present invention is to propose a culture substrate for methanisation and its method for preparing, which are inexpensive and they are environmentally friendly.

The object of the present is to respond at least partially to the aforementioned objects by proposing a more effective culture substrate. To this effect, it proposes a culture substrate intended for being used in a method for methanising liquid effluents with structured packing, formed by more than 50% of wood elements of which at least one dimension is greater than 80 mm.

Thanks to these arrangements, an effective and sustainable culture substrate for methanisation can be obtained simply and inexpensively, with a material that is environmentally friendly.

According to other characteristics:
the porosity of the culture substrate can be greater than 50%, which improves the effectiveness of the methanisation method,
said wood elements may comprise branched and/or skewed branches, these elements being easy to find and making it possible to reclaim the green waste,

The present invention also relates to a method for preparing a culture substrate according to the invention, comprising in order the following steps:
collecting green waste,
inserting green waste into a methanisation unit so as to form a structured packing.

Thanks to these arrangements, an effective culture substrate for methanisation can be obtained simply and inexpensively, with a material that is environmentally friendly, while still reclaiming the green waste.

According to other characteristics:
the method for preparing may comprise a grinding step after the collecting step and before the insertion step, the grinding being a slow grinding, so as to obtain that a majority of the ground elements have a largest dimension that is less than a metre, which makes it possible to no longer have elements that are excessively large to integrate the culture substrate,
the method for preparing may comprise a screening step before the insertion step, the screening being done at a dimension comprised between 30 mm and 80 mm, which makes it possible to eliminate the fine particles of green waste that would not be stable by methanisation, which would reduce the porosity of the culture substrate and that would risk clogging it,
the method for preparing may comprise a composting step before the screening step, which makes it possible during screening to separate from the compost the green waste that is non-degradable by methanisation, in particular wood, which will form a stable culture substrate over time.

The present invention also relates to a methanisation method comprising in order the following steps:
inserting a culture substrate according to the invention into an upstream methanisation tunnel,
sending liquid effluents into the upstream methanisation tunnel,
inserting a culture substrate according to the invention into a downstream methanisation tunnel,
sending effluents treated at the output of the upstream methanisation tunnel into the downstream methanisation tunnel.

The sequential operation offset over time of the method allows for a continuous methanisation of the liquid effluents, even when one of the tunnels is stopped. In addition the inoculation of the nouveau substrate by the effluent treated on the substrate during operation allows for a very fast colonisation of the culture substrates, by placing the tunnel having a new culture substrate downstream from a tunnel already in operation.

According to other characteristics:
a portion comprised between 30% and 60% of the effluents treated at the output of the downstream methanisation tunnel can be sent back as input of the upstream methanisation tunnel, which allows for a dilution of the effluents before the methanisation thereof and/or an inoculation of the new substrate, and thus a faster methanisation.

The present invention will be understood better when reading the following detailed description, in reference to the accompanying figures wherein:

is a schematic view of a wood element of a culture substrate according to a preferred embodiment of the invention.

is a schematic view of a wood element of a culture substrate according to a second embodiment of the invention.

is a schematic view of a wood element of a culture substrate according to a third embodiment of the invention.

is a schematic view of a methanisation installation according to a preferred embodiment of the invention.

The culture substrate 1 according to the invention is intended for being used in a method for methanising liquid effluents with structured packing. The culture substrate 1 is made up of more than 50% of wood elements 2 of which at least one dimension is greater than 80 mm.

The dimension of the wood elements 2 allows the culture substrate 1 to have a high porosity, which favours the flow of the fluids and the obtaining of a high exchange surface for bacterial development on the one hand, and between the culture substrate and the liquid effluents to be treated on the other hand. This porosity is further improved by using wood elements of diverse and irregular shapes. If the elements are of smaller dimensions, they nest together more easily by leaving little space between them, which results in most cases in a culture substrate 1 that does allow for a sufficient flow of the fluids. This can cause a compaction of the substrate, even an occlusion, reducing the exchange surface.

In the present invention, the term “porosity”, applied to a culture substrate, means the fraction of volume occupied by air in the total volume of the culture substrate.

The measurement of the porosity can for example be carried out in the following way:
the culture substrate 1 is disposed in a container,
the container is filled with water, quickly enough so that the water does not have the time to infiltrate inside the elements comprising the culture substrate 1, in particular the wood elements 2, so that the porosity internal to these elements is not taken into account,
the porosity is then equal to the volume of water added in the container, divided by the volume of the container.

The porosity of the culture substrate 1 is preferably greater than 50%, which allows for an exchange surface that is satisfactory for the effectiveness of the methanisation method between on the one hand the culture substrate 1 and the bacteria that it hosts, and on the other hand the liquid effluents. Such a porosity also makes it possible to reduce the risks of the culture substrate 1 being obstructed or clogged by biological and/or mineral deposits, requiring cleaning operations or replacement of the culture substrate 1. A longer service life of the culture substrate 1 is thus obtained.

A higher porosity allows, up to a certain point, the maintaining of a sufficient flow with the maintaining of the exchange surface, or surface available for the development of bacteria, which allows for a greater effectiveness of the methanisation reaction. It is indeed the maintaining of the exchange surface that allows for the greatest effectiveness, but it is very difficult to measure; that is why the porosity is used to characterise an effective culture substrate.

Examples of measured porosities give values of 63%, 65%, and 70%. These are effectively values that are on the average higher than for wood chips, where the porosity is generally comprised between 40% and 60%.

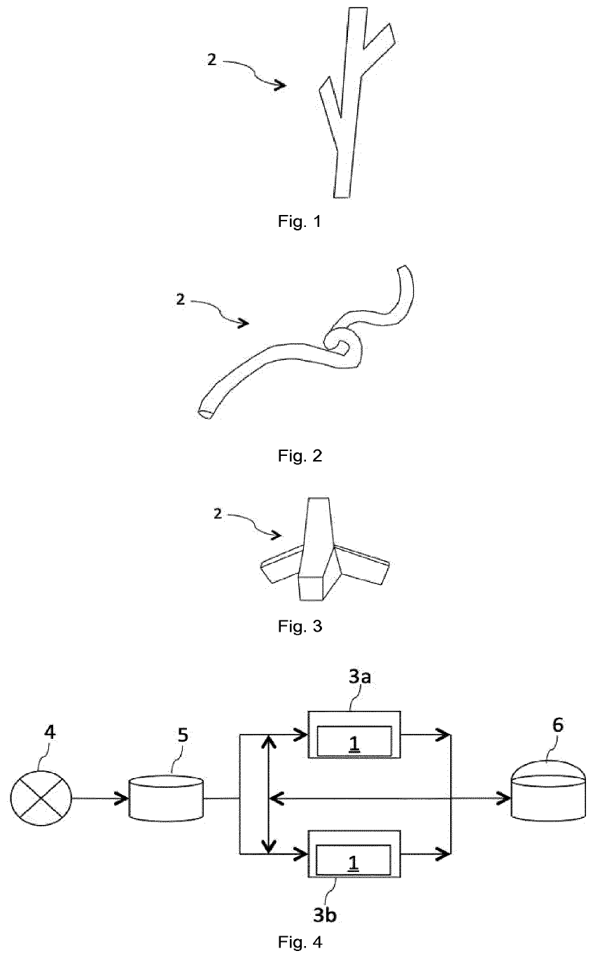

In a preferred embodiment of the invention, the wood elements 2 are branched and/or skewed branches. In the scope of the present invention, the expression “branched branch” designates a branch including at least one ramification, i.e. it includes at least two linear parts, not necessarily straight, forming an angle between them. An example is shown in FIG. 1. The expression “skewed branch” designates a branch of which the shape is such that it is not contained in a plane. An example is shown in FIG. 2.

The wood elements 2 of the branched and/or skewed branch type have the advantage when they are superposed, of leaving free spaces between them, so that a higher global porosity of the culture substrate 1 and a higher exchange surface are obtained.

They are moreover easy to find and inexpensive: this can be for example green waste formed mostly of wood, or the non-degraded portion of a composting of green waste, that can be recovered at the output of the composting. This can be for example screening residues of the compost.

In the present invention, the term “green waste” designates all of the materials coming from ligno-cellulosic plants and coming from trimming, cutting or the maintenance of these plants, for example during the maintaining of gardens, green areas, forests, hedges, or trees. The green waste can also include the by-products coming from the transformation and the reclaiming of wood, a by-product being a substance or an object coming from a production process of which the first purpose is not the production of this substance or this object.

Other types of wood elements 2 can be used, as long as their shape provides a substantial porosity to the culture substrate and a good exchange surface. This can be for example elements in the form of a tetrapod, as shown in FIG. 3, forestry chips, shreddings, or of yet another form.

The culture substrate 1 according to the invention can be prepared by implementing a method comprising in order the following steps:
collecting green waste,
inserting green waste into a methanisation unit so as to form a structured packing.

The culture substrate 1 is thus obtained inexpensively. In addition it is obtained locally, and allows for a reclaiming of this waste.

A grinding step can be implemented between the collection step and the insertion step. The grinding is then a slow grinding, so as to obtain that a majority of the ground elements have a largest dimension that is less than a metre. This grinding can be necessary if the green waste collected is too large, which can cause a problem for the insertion step, or which can result in an excessively porous culture substrate 1, the presence of grosses branches preventing the introduction of thinner branches. An excessively porous culture substrate 1, for example greater than 90%, results in a reduced exchange surface between the effluents to be treated and the bacteria, and therefore degrades the performance of the methanisation method.

A screening step can be implemented before the insertion step, after the grinding if there is one. The screening, which can be done for example at a dimension comprised between 30 mm and 80 mm, makes it possible to get rid of the fine particles that would participate in the occlusion of the culture substrate, which would reduce the effectiveness of the method of methanisation.

A composting step can take place before the screening step. This makes it possible to degrade the green waste so that once the compost is eliminated by the screening step, there only remains the material that is not degradable by compost, rich in lignin. As these materials rich in lignin are not, or are very little, degradable by methanisation, they make it possible to obtain a stable culture substrate 1.

The culture substrate 1 can thus be obtained by recycled materials, treated if necessary by conventional steps of treating green waste. The culture substrate 1 is thus inexpensive, obtained locally and is environmentally friendly, and makes it possible to obtain good performance when it is used in a methanisation method.

The culture substrate 1 can be used in a continuous methanisation method, shown in FIG. 4 and comprising in order the following steps:
inserting a culture substrate 1 into an upstream methanisation tunnel 3a,
sending liquid effluents into the upstream methanisation tunnel 3a,
inserting a culture substrate 1 into a downstream methanisation tunnel 3b,
sending effluents treated at the output of the upstream methanisation tunnel 3a at the input of the downstream methanisation tunnel 3b.

Since it was set into operation, the culture substrate 1 of the upstream methanisation tunnel 3a has had the time to be colonised by bacteria. The effluents sent into the downstream methanisation tunnel 3b are then charged with bacteria, and the colonisation of the downstream methanisation tunnel 3b is very fast, which improves the performance of the method of methanisation.

When the upstream methanisation tunnel 3a is at the end of its life, the downstream methanisation tunnel 3b, of which the culture substrate 1 is younger, continues to operate alone, the time to replace the culture substrate in the first methanisation tunnel 3a. The downstream methanisation tunnel 3b then becomes the upstream methanisation tunnel 3a, and vice versa. The method is thus repeated continuously, each methanisation tunnel 3a, 3b having in turn a culture substrate 1 that is older than the other methanisation tunnel 3b, 3a, with the methanisation tunnel 3a, 3b having the oldest culture substrate being placed upstream from the other methanisation tunnel 3b, 3a.

Using two methanisation tunnels 3a, 3b makes it possible to have a method that operates without interruption, not only during the end of the life of one of the methanisation tunnels 3a, 3b, but also during maintenance operations or when undesirable items are removed from one of the methanisation tunnels 3a, 3b.

In a preferred embodiment of the invention, a portion comprised between 30% and 60% of the effluents treated at the output of the downstream methanisation tunnel 3b are sent back into the upstream methanisation tunnel 3a. This allows for a dilution of the effluents as input, which are sometimes excessively thick to allow for a fast and effective methanisation.

Such a dilution can also be implemented when the upstream methanisation tunnel 3a operates alone, in particular before the implementation of the downstream methanisation tunnel 3b. There is then a portion comprised between 30% and 60% of effluents treated at the output of the upstream methanisation tunnel 3a sent back as input of the upstream methanisation tunnel 3a.

As shown in FIG. 4, before the methanisation in the methanisation tunnels 3a, 3b the method of methanisation according to the invention may comprise one of the following two steps:
preparing in a deconditioner and/or grinder 4,
hygienising in a hygienisation unit 5.

After methanisation, the biogas can be the object of post-storage in a gasometer 6.

The method for methanisation according to the invention allows the retention time of the solids (SRT) in the methanisation tunnels 3a, 3b to be maximised. Thus an optimum colonisation of the substrate is obtained and the conservation of the bacteria of interest by decoupling the retention time of the liquid effluents (biowaste) by that of the solids (bacteria/culture substrate).

Although the description hereinabove is based on particular embodiments, it in no way limits the scope of the invention, and modifications can be made, in particular by substitution of technical equivalents or by different combinations of all or a part of the characteristics developed hereinabove.

1. A methanisation unit comprising:
a culture substrate intended for being used in a method for methanising liquid effluents with structured packing, said culture substrate being made up of more than 50% of wood elements of which at least one dimension is greater than 80 mm, the porosity of said culture substrate being greater than 50%.

2. The methanisation unit according to claim 1 wherein said wood elements comprise branched and/or skewed branches.

3. A method for preparing a culture substrate intended for being used in a methanisation unit according to claim 1, comprising in order the following steps:
collecting green waste,
inserting green waste into said methanisation unit so as to form a structured packing.

4. The method for preparing according to claim 3, further comprising a grinding step after the collecting step and before the insertion step, the grinding being a slow grinding, so as to obtain that a majority of the ground elements have a largest dimension that is less than a metre.

5. The method for preparing according to claim 3, comprising a screening step before the insertion step, the screening being done at a dimension comprised between 30 mm and 80 mm.

6. The method for preparing according to claim 5, comprising a composting step before the screening step.

7. A method for methanising in a methanisation unit according to claim 1.

8. The method for methanising according to claim 7, wherein said culture substrate is prepared by the following steps in order:
collecting green waste; and
inserting green waste into said methanisation unit so as to form a structured packing.

9. The method for methanising according to claim 7 comprising in order the following steps:
inserting a culture substrate into an upstream methanisation tunnel (3a),
sending liquid effluents into the upstream methanisation tunnel,
inserting a culture substrate into a downstream methanisation tunnel,
sending effluents treated at the output of the upstream methanisation tunnel into the downstream methanisation tunnel.

10. The method for methanising according to claim 7, wherein a portion comprised between 30% and 60% of the effluents treated at the output of the downstream methanisation tunnel is sent back as input of the upstream methanisation tunnel.
